(19) United States
(12) Patent Application Publication
(10) Pub. No.: US 2026/0339995 A1
(43) Pub. Date: Oct. 29, 2026

(54) MULTI-MODAL LANGUAGE MODELS FOR HEALTH GROUNDED IN PATIENT SPECIFIC-FEATURES

(71) Applicant: GOOGLE LLC, Mountain View, CA (US)

(72) Inventors: Nicholas A. Furlotte, San Francisco, CA (US); Cory Yuen Fu McLean, Newton, MA (US); Justin Thomas Cosentino, San Francisco, CA (US); Farhad Hormozdiari, Cambridge, MA (US); Anastasiya Belyaeva, San Francisco, CA (US)

(21) Appl. No.: 19/487,258

(22) PCT Filed: May 28, 2024
(86) PCT No.: PCT/US2024/031299

§ 371 Date: Nov. 24, 2025

Related U.S. Application Data
(60) Provisional application No. 63/504,424, filed on May 25, 2023.

(51) Int. Cl. G06F 16/3329; G06F 40/284; G06N 3/0455; G16H 50/30

(52) U.S. Cl. CPC G06F 16/33295; G06F 40/284; G06N 3/0455; G16H 50/30

(57) ABSTRACT

Methods, systems, and apparatus, including computer programs encoded on computer storage media, for generating responses to health-related queries using a language model neural networks. In particular, a multi-modal language model neural network is used to process a query relating to a given patient and multi-modal features relating to the given patient to generate a response to the query.

### CROSS-REFERENCE TO RELATED APPLICATIONS

This application claims the benefit of priority to U.S. Provisional Patent Application No. 63/504,424, filed on May 25, 2023, the contents of which are hereby incorporated by reference.

### BACKGROUND

This specification relates to processing inputs using neural networks.

Neural networks are machine learning models that employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current value inputs of a respective set of parameters.

### SUMMARY

This specification describes a system implemented as computer programs on one or more computers in one or more locations that generates responses to queries relating to the health of a particular patient.

Particular embodiments of the subject matter described in this specification can be implemented so as to realize one or more of the following advantages.

Foundation large language models (LLMs) have shown an impressive ability to solve tasks across a wide range of fields. However, pre-trained LLMs by default struggle to solve personalized health tasks, i.e., tasks that require responding to queries about the health of a particular patient given patient-specific information for the patient.

To effectively solve personalized health tasks, LLMs need the ability to ingest a diversity of data modalities that are relevant to an individual's health status.

This specification describes techniques for generating a multimodal LLM for health that is grounded in individual-specific data across multiple modalities by representing the multiple modalities in the same natural language embedding space used by the LLM. Thus, the described techniques can make use of, e.g., a foundational LLM, without needing to further train the LLM in order to effectively adapt the LLM for use in personalized health settings.

Moreover, the same LLM can be used to respond to queries relating to multiple conditions, allowing a single model to be used rather than requiring specialized models for each condition. As a result, the system that deploys the LLM is more computationally-efficient, i.e., as compared to a system that requires separate neural networks for each condition of interest. Moreover, after training, the resulting multi-modal LLM can generalize to conditions that were not included in the training data set used to train the modality-specific encoder neural networks that are used in adapting the LLM.

The details of one or more embodiments of the subject matter of this specification are set forth in the accompanying drawings and the description below. Other features, aspects, and advantages of the subject matter will become apparent from the description, the drawings, and the claims.

### BRIEF DESCRIPTION OF THE DRAWINGS

FIG. 1 shows an example neural network system.

FIG. 2 shows the operation of the system.

FIG. 3 is a flow diagram of an example process for generating a response to a query.

FIG. 4 is a flow diagram of an example process for training the modality-specific encoder neural networks.

FIG. 5 is an example of the performance of the described techniques.

Like reference numbers and designations in the various drawings indicate like elements.

### DETAILED DESCRIPTION

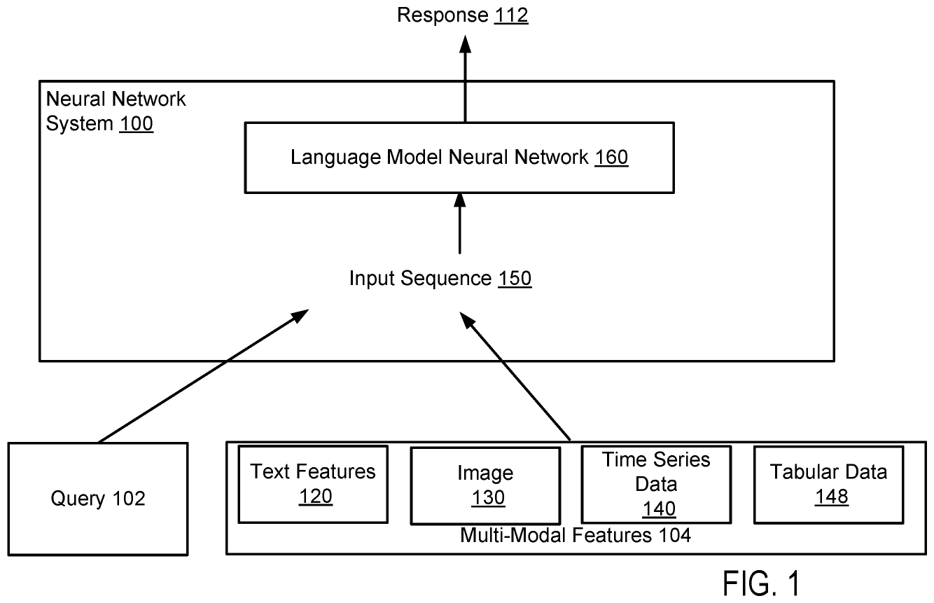

FIG. 1 shows an example neural network system 100. The neural network system 100 is an example of a system implemented as computer programs on one or more computers in one or more locations, in which the systems, components, and techniques described below can be implemented.

The system 100 generates responses 112 to queries 102, where each query 102 relates to the health of a respective patient.

In particular, for any given patient, the responses 112 generated by the system 100 are conditioned on multi-modal health features 104 associated with the patent.

The multi-modal health features 104 for a given patient include one or more features from each of a plurality of modalities. That is, the features for any given patient include features from multiple different modalities.

For example, the multiple modalities can include a text feature modality 120, e.g., text features that are generated from one or more of demographic information for the patient, clinical information for the patient, and so on. As a particular example, the text features can be generated by serializing a tabular representation of various information about the patient.

As another example, the multiple modalities can include one or more image modalities 130. i.e., one or more modalities that represent features as images. Images may be captured of the physical environment of the patient. e.g., which may include, for example, part or whole of the body of the patient. As one example, a given image modality can represent a particular type of medical image of a portion of the body of the patient.

As another example, the multiple modalities can include one or more time series data modalities 140, i.e., one or more modalities that represent features as a time series. These may be features which reflect physical measurements of physical characteristics across time. As one example, a given time series modality can represent results across time of a particular type of laboratory test or diagnostic measurement for the patient. As a specific example of this, the time series data modality can include a spirogram, which is a time-series curve that measures the amount of air an individual can breath in and out of their lungs. Spirometry is commonly used to assess pulmonary function and the presence of respiratory diseases, but cannot by default be represented as text.

As another example, the multiple modalities can include one or more tabular modalities 148, e.g., one or more modalities that represent features as a table of rows and columns. Such features may represent measurements of physical characteristics, such as measurements of characteristics relating to the body of the patient. As one example, a given tabular modality can represent various results of a particular type of laboratory test for the patient as a table.

Generally, the multiple different modalities can represent any of a variety of information relating to the patient, e.g., information such as lab values (e.g., cholesterol and triglycerides), health tracker metrics (e.g., daily step count and heart rate), genome sequences, genetic risk scores, and other omics data modalities. This information may reflect measurements taken of the patient or their environment.

To generate the response, the system 100 generates a respective representation of each of the one or more features from each of the plurality of modalities as a respective set of one or more tokens in a natural language embedding space.

That is, the system 100 represents each feature of each modality as a respective set of one or more tokens in the natural language embedding space, even if features of the modality are not represented as natural language.

For example, as will be described in more detail below, for one or more of the modalities, the system 100 can process the features of the modality using an encoder neural network that is specific to the modality to generate the tokens in the natural language embedding space that represent the feature.

The system 100 then generates an input sequence 150 of tokens that includes one or more tokens in the natural language embedding space representing the query 102 and the respective sets of one or more tokens in the natural language embedding space that represent each of the one or more features from each of the plurality of modalities.

Thus, although the other modalities are not natively represented as natural language text, the system 100 nonetheless jointly represents both the (natural language) query 102 and the multi-modal features 104 in a single sequence 150 of tokens in the natural language embedding space.

The system processes the input sequence 150 of tokens using a language model neural network 160 to generate a language model output 170 and generates, from the language model output, a response 112 to the query 102.

Generally, the language model neural network 160 is a neural network that processes an input sequence of tokens in the natural language embedding space to generate an output sequence that represents output tokens in the natural language embedding space. For example, the output tokens in the natural language space can correspond to text tokens, e.g., any of characters, word pieces, byte pair encodings of text, words, and so on. In some implementations, the language model neural network can also generate output tokens that correspond to features in non-text modalities, e.g., any of the modalities described above.

As one example, the language model neural network 160 can be a large language model (LLM), e.g., an LLM with a decoder-only Transformer architecture. As another example, the language model neural network 160 can be a visual language model (VLM).

Example architectures of the language model neural network 160 as well as techniques for training such a neural network are described in more detail in Chowdhery, A., et al., Palm: Scaling language modeling with pathways, arXiv preprint arXiv:2204.02311, 2022, Kojima, T., et al., Large language models are zero-shot reasoners, arXiv preprint arXiv:2205.11916, 2022, Alayrac, J. B., et al. Flamingo: a visual language model for few-shot learning, arXiv preprint arXiv:2204.14198, 2022, Wei, et al, Fine-tuned Language Models Are Zero-Shot Learners, arXiv preprint arXiv:2109.01652, 2022, and so on.

As a particular example, the language model neural network 160 can be configured to autoregressively generate an output sequence of text tokens (and, optionally, other types of tokens) conditioned on the input sequence 150.

In particular, to generate the output sequence, the language model neural network 160 is configured to, for each position in the output sequence, generate a probability distribution over a vocabulary of text tokens conditioned on the input sequence and any text tokens at positions preceding the position in the output sequence and then select, using the probability distribution, a text token from the vocabulary of text tokens as the text token at the position.

In some cases, the response 112 is a natural language sequence that is generated by the language model neural network 160.

In some other cases, the response 112 is a score that is computed based on output of the language model neural network or a likelihood derived from the score. For example, the score that is output can be a probabilistic classification of the data reflected across multiple modalities. Moreover, this classification may be directed towards the query, i.e., so that the score is a probabilistic classification of the multiple modalities of data, with the class to which the probability is assigned being defined by the query.

For example, the score can indicate one of the likelihood that the patient has a particular medical condition specified in the query, the likelihood that the patient will suffer a particular adverse health event specified in the query within a threshold amount of time, the risk level of the patient developing the particular medical condition specified in the query, the risk level of the patient suffering a particular adverse health event specified in the query within a threshold amount of time, and so on.

Once generated, the system 100 can provide the response 112, e.g., for presentation to the particular patient or to a clinician treating the particular patient.

For example, the system 100 can be implemented as part of a user-facing health and wellness chatbot and the queries 102 can be received as input by the system 100 from users. By conditioning the chatbot on the multi-modal user features, the chatbot can provide responses 112 that are specific to the current user rather than generalized responses that may not be relevant to the current user based on the user's current health status.

As another example, the system 100 can be implemented as part of a clinician assistant software system and the queries 102 can be received as input by the system 100 from clinicians or automatically generated by another component of the clinician software. By conditioning the clinician assistant on the multi-modal user features, the clinician assistant can provide responses 112 that are specific to the current patient rather than generalized responses that may not be relevant to the current patient based on the patient's current health status.

As yet another example, the system 100 can be implemented as part of an early warning software system employed by a medical treatment facility, e.g., a hospital or other medical treatment facility, that generate suggestions for escalating patient care by predicting which patients conditions are likely to clinically deteriorate. Thus, the queries 102 may be generated automatically by the system 100 whenever certain criteria are met, e.g., when a given patient is admitted, when a certain amount of time has passed since the given patient was last evaluated, when a new diagnostic or lab test result is available and so on. By conditioning the responses 112 on the multi-modal user features, the early warning software system can provide responses 112 that are specific to the current patient rather than generalized responses that may not be relevant to the current patient based on the patient's current health status.

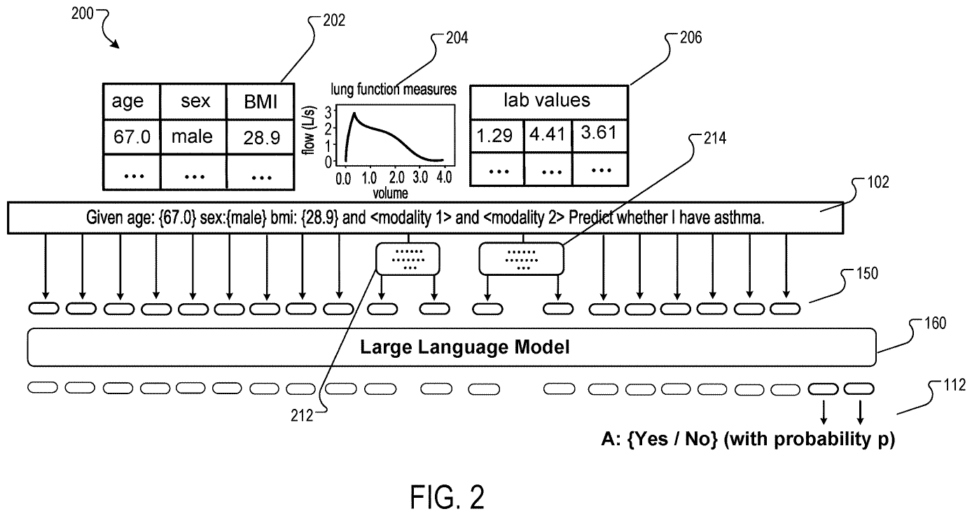

FIG. 2 shows an example 200 of the operation of the system 100.

In the example 200, the system 100 receives a query 102: “Predict whether I have asthma.”

The system 100 also receives multi-modal features 104 that include a first table 202 that represents patient information (age, sex, and BMI (body mass index)), time series data 204 that represents the lung function of the patient, and a second table 206 that represents clinical data, e.g., lab results, for the patient.

The system 100 generates a respective representation of each of the multi-modal features 104 as a respective set of one or more tokens in the natural language embedding space.

In the example 200, the system generates, from the first table 202 that represents patient information (age, sex, and BMI (body mass index)), a serialized natural language input by mapping table column names and corresponding values into a structured format and then serializing the names and values in the structure format into a natural language sequence. The system then generates tokens in the embedding space by tokenizing the serialized natural language input.

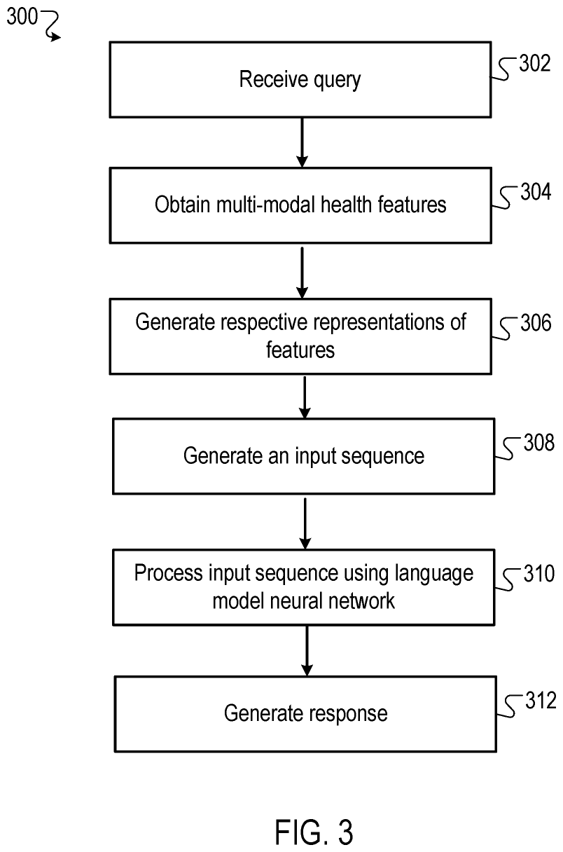

For the time series data 204 that represents the lung function of the patient and the second table 206 that represents clinical data for the patient, the system 100 processes these using respective modality-specific encoder neural networks 212 and 214 in order to generate the tokens in the natural language space that represent these features. Processing inputs using encoder neural networks will be described in more detail below with reference to FIG. 3.

The system 100 then generates an input sequence 220 of tokens that includes one or more tokens in the natural language embedding space representing the query 102 and the respective sets of one or more tokens in the natural language embedding space that represent each of the one or more features from each of the modalities.

The system then processes the input sequence 220 of tokens using the language model neural network 160 to generate a language model output and, generates, from the language model output, a response 112 to the query 102.

In the example 200, the response 112 can either indicate “yes” or “no” and give an associated probability based on the score that is assigned by the language model output to a predetermined set of tokens, e.g., the set of one or more tokens representing “yes” or the set of one or more tokens representing “no.”

In other examples, the response 112 can be a natural language output sequence, e.g., “You have asthma with probability x.”

FIG. 3 is a flow diagram of an example process 300 for generating a response to a query. For convenience, the process 300 will be described as being performed by a system of one or more computers located in one or more locations. For example, a neural network system, e.g., the neural network system 100 of FIG. 1, appropriately programmed, can perform the process 300.

The system receives a query relating to a health of a particular patient (step 302).

The system obtains multi-modal health features associated with the particular patent (step 304).

As described above, the multi-modal health features include one or more features from each of a plurality of modalities. Such features may be obtained by one or more independent processes, and may represent physical characteristics, such as characteristics of the body of the patient, and/or other information. In some examples, multiple modalities relate to physical characteristics; in such circumstances multiple modalities may represent data relating to the same physical characteristics or to different characteristics.

The system generates a respective representation of each of the one or more features from each of the plurality of modalities as a respective set of one or more tokens in a natural language embedding space (step 306).

That is, each token is an embedding. i.e., an ordered collection of numerical values, having a fixed number of values, i.e., a fixed dimensionality. The embedding space is the space of embeddings that have the fixed number of values.

The embedding space is referred to as “a natural language embedding space” because it is the same space as the space of text tokens that are processed by the language model neural network. In other words, the tokens representing the multi-modal features each have the same dimensionality as the text tokens that are processed by the language model neural network.

The way in which the system generates the tokens in the natural language embedding space depends on the modality to which the features belong.

For example, when the multi-modal health features include text features, the system can generate, from the text features, a natural language text sequence.

The system then tokenizes the natural language sequence into a plurality of text tokens. e.g., by applying a text tokenizer to the natural language sequence, i.e., the SentencePiece tokenizer or another appropriate tokenizer.

The system then maps each text token to a respective token in the natural language embedding space using a text embedding matrix, e.g., either the same text embedding matrix used by the language model neural network to map text tokens or a separately learned text embedding matrix that is only used for text features.

As one example, the text features can be tabular features, so that each text feature has a corresponding feature name and a feature value.

In this case, the system can serialize the text features into a structured format and then generate the natural language sequence that represents the text features in the structured format. That is, the system can serialize the corresponding feature names and the feature values into the natural language sequence.

As another example, for each modality in a set of one or more of the modalities and for each of the one or more features of the modality, the system can process the feature using an encoder neural network specific to the modality to generate one or more tokens in the natural language embedding space that represent the feature.

That is, because the encoder neural networks are specific to their corresponding modalities, when the set of modalities includes two or more modalities, the respective encoder neural networks that are specific to the two or more modalities are different from one another. That is, when the set of modalities for which the system maintains encoder neural networks includes more than one modality, the system processes each modality in the set using a different, modality-specific encoder neural network.

As one example, the set of modalities can include an image modality and the encoder neural network that is specific to the modality can be an image embedding neural network. For example, the image embedding neural network can be a convolutional neural network or a Vision Transformer neural network.

As another example, the set of one or more modalities can include a tabular data modality and the encoder neural network that is specific to the modality can be a tabular data embedding neural network. For example, the one or more features of the tabular data modality can be laboratory test results for the particular patient. The tabular data modality embedding neural network can have any appropriate architecture that allows the neural network to map a representation of the tabular data to a set of one or more tokens in the embedding space. As one example, the tabular data embedding neural network can be a multi-layer perceptron (MLP).

As another example, the set of one or more modalities can include a time series data modality, with the encoder neural network that is specific to the modality being a time series encoder neural network.

For example, the features of the tabular data modality can each be a respective a time series representing diagnostic measurements for the particular patient across time.

The time series encoder neural network can have any appropriate architecture that allows the neural network to map a representation of the time series to a set of one or more tokens in the embedding space. For example, the time series encoder neural network can be a 1-D convolutional neural network, a recurrent neural network, or a Transformer neural network.

The system generates an input sequence of tokens in the natural language embedding space (step 308).

The sequence includes one or more tokens representing the query and the respective sets of one or more tokens in the natural language embedding space that represent each of the one or more features from each of the plurality of modalities.

For example, the system can tokenize the query into a plurality of text tokens, e.g., by applying a text tokenizer to the natural language sequence, i.e., the SentencePiece tokenizer or another appropriate tokenizer and then map each text token to a respective token in the natural language embedding space using a text embedding matrix.

The system can then combine the one or more tokens representing the query and the respective sets of one or more tokens in the natural language embedding space that represent each of the one or more features from each of the plurality of modalities according to a pre-defined format to the generate the input sequence.

Thus, each token in the input sequence is either (i) a token in the natural language embedding space representing a text token and generated by applying the text embedding matrix to the text token or (ii) a token in the natural language embedding space representing a feature of a non-text modality and generated by applying a modality-specific encoder neural network for the corresponding non-text modality to the feature.

As a particular example, when the inputs include (i) text tokens, e.g., from the query, from features of the text modality, or both, (ii) one or more features from the time series modality, and (iii) one or more features from the tabular modality, any given token xᵢ at aby given position i in the input sequence can be represented as:

$${x}_{i} = \{ \begin{matrix}γ ( {w}_{i} ) & if i is a text token \\ {ϕ}_{s} ( {s}_{i} ) & if i corresponds to the time series modality \\ {ϕ}_{t} ( {t}_{i} ) & if i corresponds to the tabular modality\end{matrix}$$

where wᵢ is a text token and γ represents the text embedding matrix, sᵢ is a time series feature and φₛ s represents the time series encoder, tᵢ is a tabular feature and φₜ represents the tabular data encoder.

The system processes the input sequence of tokens using a language model neural network to generate a language model output (step 310).

Generally, the language model neural network is configured to autoregressively generate an output sequence of text tokens conditioned on the input sequence. In particular, the language model neural network is configured to for each position in the output sequence, generate a probability distribution over a vocabulary of text tokens conditioned on the input sequence and any text tokens at positions preceding the position in the output sequence and then select, using the probability distribution, a text token from the vocabulary as the text token at the position in the output sequence.

Depending on the type of response required, the language model output can either be an output sequence or can be a score, e.g., a log likelihood, assigned by the language model to a pre-determined output sequence.

The system generates, from the language model output, a response to the query (step 312).

As one example, the response to the query is a score that indicates a likelihood that the particular patient has a medical condition specified in the query.

In this example, the system processes the input sequence of tokens using the language model neural network to generate a language model output to determine a respective probability assigned by the language model neural network to each text token from a pre-determined output sequence of one or more text tokens conditioned on the input sequence. The system then generates the response by generating the score from the respective probability assigned by the language model neural network to each text token from a pre-determined output sequence of one or more text tokens. For example, the score can be the log likelihood of the respective probabilities.

As another example, the response to the query can be a natural language response to the query.

In this example, the system processes the input sequence of tokens using the language model neural network to generate one or more candidate output sequences of text tokens, and then provides, as the response, one of the candidate output sequences of text tokens generated by the language model neural network. For example, the system can generate a single output sequence by greedily selecting the highest probability text tokens. As another example, the system can generate multiple candidate output sequences by performing a beam search and then select, as the candidate output sequence to provide as the response, the candidate output sequence that has the highest log likelihood.

In some cases, the system can generate, from the response, a further input to another neural network, e.g., another language model neural network, and use the other neural network to augment the final response. For example, when the response is a score that indicates a likelihood that the particular patient has a medical condition specified in the query, the system can generate another input sequence that includes the score and provide the other input sequence as input to the other neural network to obtain, as output, additional information relating to the medical condition.

For example, when the condition is asthma, the system can generate a text sequence “I have a p % chance of having asthma. What are some exercises you would suggest, given this information? Please tailor your recommendations to my health status.” The system can then process this text sequence using another LLM to obtain exercise recommendations, and provide these exercise recommendations along with the score as part of the final response.

Prior to using the modality-specific neural networks when responding to queries, the system trains the modality-specific neural networks.

In particular, prior to being used by the system, the language model neural network has been pre-trained on a language modeling task. Optionally, as part of the pre-training, the language model neural network can have been pre-trained on one or more additional tasks, e.g., supervised fine-tuning, instruction tuning, and so on. After the language model neural network has been pre-trained, the system trains the encoder neural networks on a task that measures responses to health-related queries generated by the language model neural network. During this training, the system holds the language model neural network frozen.

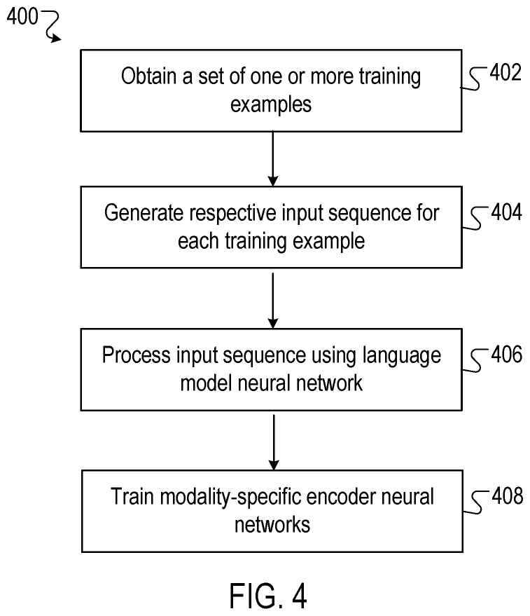

An example of this training is described below with reference to FIG. 4.

Thus, the system does not need to train the language model neural network and only needs to train the modality-specific encoder neural networks in order yield a multi-modal language model that can effectively respond to health-related queries by leveraging multi-modal features.

FIG. 4 is a flow diagram of an example process 400 for training modality-specific encoder neural networks. For convenience, the process 400 will be described as being performed by a system of one or more computers located in one or more locations. For example, a neural network system, e.g., the neural network system 100 of FIG. 1, appropriately programmed, can perform the process 400.

The system can repeatedly perform the process 400 to train the modality-specific encoders for each of the modalities.

The system obtains a set of one or more training examples (step 402). Each training example includes a query, corresponding multi-modal features, and a ground truth response. In some cases, the queries can include queries about multiple different conditions, i.e., rather than each query being constrained to relate to the same condition.

The system generates a respective input sequence for each training example (step 404), e.g., using the modality-specific encoders as described above with reference to FIGS. 2 and 3.

The system processes the input sequence using the language model neural network to generate, for each training example, a respective likelihood for each token in the ground truth response in the training example (step 406).

The system trains the modality-specific encoder neural networks on a loss function that measures, for each training example, the respective likelihoods for the tokens in the ground truth response in the training example (step 408).

That is, the system can determine, for each encoder, a respective gradient with respect to the parameters of the encoder by backpropagating gradients of the loss function through the language model neural network and then apply an optimizer, e.g., Adam, rmsProp, AdamW, Adafactor, and so on, to the respective gradient to update the parameters of the encoder. During this training, the system holds the language model neural network fixed, i.e., to the pre-trained values described above.

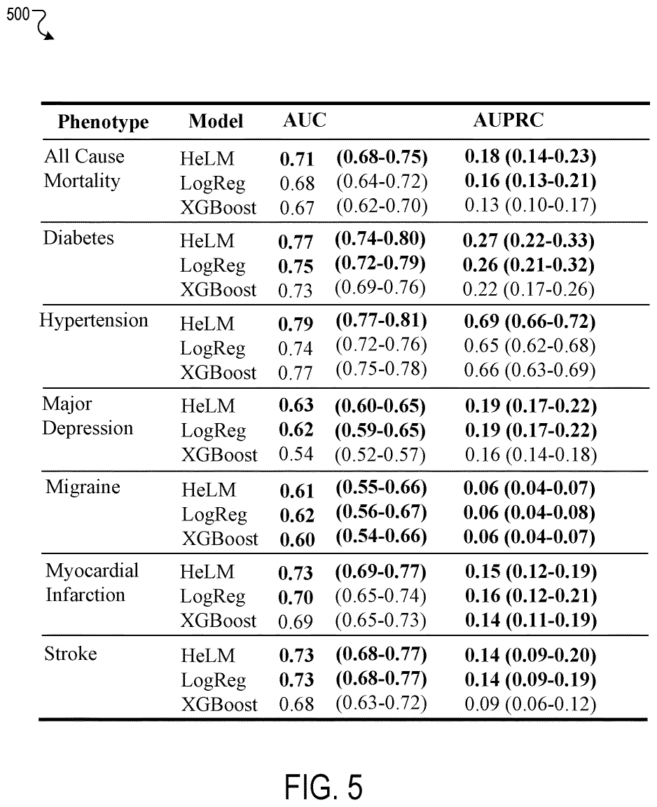

FIG. 5 shows an example 500 of the performance of the described techniques.

In particular, the example 500 shows the performance of the described techniques (“HeLM”) on queries for multiple different conditions relative to 1) a baseline technique that trains a respective logistic regression classifier (“LogReg”) for each condition and 2) another baseline technique that trains an XGBoost classifier for each condition.

As can be seen from the example 500, the described techniques achieve comparable and, in many cases, better performance, despite the fact that the single model was trained for multiple conditions and the baselines use different, specifically-trained models for each condition.

Thus, as can be seen from the example 500, the describes techniques achieve comparable or better performance than specialized models, but can be applied to generate responses to a variety of queries and using any of a variety of multi-modal features as context.

This specification uses the term “configured” in connection with systems and computer program components. For a system of one or more computers to be configured to perform particular operations or actions means that the system has installed on it software, firmware, hardware, or a combination of them that in operation cause the system to perform the operations or actions. For one or more computer programs to be configured to perform particular operations or actions means that the one or more programs include instructions that, when executed by data processing apparatus, cause the apparatus to perform the operations or actions.

Embodiments of the subject matter and the functional operations described in this specification can be implemented in digital electronic circuitry, in tangibly-embodied computer software or firmware, in computer hardware, including the structures disclosed in this specification and their structural equivalents, or in combinations of one or more of them. Embodiments of the subject matter described in this specification can be implemented as one or more computer programs, i.e., one or more modules of computer program instructions encoded on a tangible non transitory storage medium for execution by, or to control the operation of, data processing apparatus. The computer storage medium can be a machine-readable storage device, a machine-readable storage substrate, a random or serial access memory device, or a combination of one or more of them. Alternatively or in addition, the program instructions can be encoded on an artificially generated propagated signal, e.g., a machine-generated electrical, optical, or electromagnetic signal, that is generated to encode information for transmission to suitable receiver apparatus for execution by a data processing apparatus.

The term “data processing apparatus” refers to data processing hardware and encompasses all kinds of apparatus, devices, and machines for processing data, including by way of example a programmable processor, a computer, or multiple processors or computers. The apparatus can also be, or further include, special purpose logic circuitry. e.g., an FPGA (field programmable gate array) or an ASTC (application specific integrated circuit). The apparatus can optionally include, in addition to hardware, code that creates an execution environment for computer programs, e.g., code that constitutes processor firmware, a protocol stack, a database management system, an operating system, or a combination of one or more of them.

A computer program, which may also be referred to or described as a program, software, a software application, an app, a module, a software module, a script, or code, can be written in any form of programming language, including compiled or interpreted languages, or declarative or procedural languages; and it can be deployed in any form, including as a stand alone program or as a module, component, subroutine, or other unit suitable for use in a computing environment. A program may, but need not, correspond to a file in a file system. A program can be stored in a portion of a file that holds other programs or data, e.g., one or more scripts stored in a markup language document, in a single file dedicated to the program in question, or in multiple coordinated files, e.g., files that store one or more modules, sub programs, or portions of code. A computer program can be deployed to be executed on one computer or on multiple computers that are located at one site or distributed across multiple sites and interconnected by a data communication network.

In this specification, the term “database” is used broadly to refer to any collection of data: the data does not need to be structured in any particular way, or structured at all, and it can be stored on storage devices in one or more locations. Thus, for example, the index database can include multiple collections of data, each of which may be organized and accessed differently.

Similarly, in this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

The processes and logic flows described in this specification can be performed by one or more programmable computers executing one or more computer programs to perform functions by operating on input data and generating output. The processes and logic flows can also be performed by special purpose logic circuitry, e.g., an FPGA or an ASIC, or by a combination of special purpose logic circuitry and one or more programmed computers.

Computers suitable for the execution of a computer program can be based on general or special purpose microprocessors or both, or any other kind of central processing unit. Generally, a central processing unit will receive instructions and data from a read only memory or a random access memory or both. The essential elements of a computer are a central processing unit for performing or executing instructions and one or more memory devices for storing instructions and data. The central processing unit and the memory can be supplemented by, or incorporated in, special purpose logic circuitry. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device, e.g., a universal serial bus (USB) flash drive, to name just a few.

Computer readable media suitable for storing computer program instructions and data include all forms of non volatile memory, media and memory devices, including by way of example semiconductor memory devices, e.g., EPROM, EEPROM, and flash memory devices: magnetic disks, e.g., internal hard disks or removable disks; magneto optical disks; and CD ROM and DVD-ROM disks.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device. e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's device in response to requests received from the web browser. Also, a computer can interact with a user by sending text messages or other forms of message to a personal device, e.g., a smartphone that is running a messaging application, and receiving responsive messages from the user in return.

Data processing apparatus for implementing machine learning models can also include, for example, special-purpose hardware accelerator units for processing common and compute-intensive parts of machine learning training or production, i.e., inference, workloads.

Machine learning models can be implemented and deployed using a machine learning framework, e.g., a TensorFlow framework or a Jax framework.

Embodiments of the subject matter described in this specification can be implemented in a computing system that includes a back end component, e.g., as a data server, or that includes a middleware component, e.g., an application server, or that includes a front end component, e.g., a client computer having a graphical user interface, a web browser, or an app through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (LAN) and a wide area network (WAN), e.g., the Internet.

The computing system can include clients and servers. A client and server are generally remote from each other and typically interact through a communication network. The relationship of client and server arises by virtue of computer programs running on the respective computers and having a client-server relationship to each other. In some embodiments, a server transmits data, e.g., an HTML page, to a user device, e.g., for purposes of displaying data to and receiving user input from a user interacting with the device, which acts as a client. Data generated at the user device, e.g., a result of the user interaction, can be received at the server from the device.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A computer-implemented method comprising:
receiving a query relating to a health of a particular patient;
obtaining multi-modal health features associated with the particular patent, the multi-modal health features comprising one or more features from each of a plurality of modalities;
generating a respective representation of each of the one or more features from each of the plurality of modalities as a respective set of one or more tokens in a natural language embedding space;
generating an input sequence of tokens that comprises one or more tokens in the natural language embedding space representing the query and the respective sets of one or more tokens in the natural language embedding space that represent each of the one or more features from each of the plurality of modalities;
processing the input sequence of tokens using a language model neural network to generate a language model output; and
generating, from the language model output, a response to the query.

2. The method of claim 1, wherein the multi-modal health features comprise text features, each text feature having a corresponding feature name and a feature value, and wherein generating a representation of each of the one or more features from each of the plurality of modalities as one or more tokens in the natural language embedding space comprises:
generating, from the text features, a natural language text sequence;
tokenizing the natural language sequence into a plurality of text tokens; and
mapping each text token to a respective token in the natural language embedding space using a text embedding matrix.

3. The method of claim 2, wherein generating, from the text features, a natural language text sequence comprises:
serializing the corresponding feature names and the feature values into the natural language sequence.

4. The method of claim 1, wherein generating a representation of each of the one or more features from each of the plurality of modalities as one or more tokens in the natural language embedding space comprises, for each modality in a set of one or more of the modalities:
for each of the one or more features of the modality, processing the feature using an encoder neural network specific to the modality to generate one or more tokens in the natural language embedding space that represent the feature.

5. The method of claim 4, wherein:
the language model neural network has been pre-trained on a language modeling task,
the encoder neural networks are trained on a task that measures responses to health-related queries generated by the language model neural network, and
the language model neural network is held frozen during the training of the encoder neural networks.

6. The method of claim 4, wherein the set of one or more of the modalities comprises two or more modalities and the respective encoder neural networks that are specific to the two or more modalities are different from one another.

7. The method of claim 4, wherein the set of one or more modalities comprises an image modality and the encoder neural network that is specific to the modality is an image embedding neural network.

8. The method of claim 4 wherein the set of one or more modalities comprises a tabular data modality and the encoder neural network that is specific to the modality is a tabular data embedding neural network.

9. The method of claim 8, wherein one or more features of the tabular data modality are laboratory test results for the particular patient.

10. The method of claim 8, wherein the tabular data embedding neural network is a multi-layer perceptron (MVLP).

11. The method of claim 4 wherein the set of one or more modalities comprises a time series data modality and the encoder neural network that is specific to the modality is a time series encoder neural network.

12. The method of claim 11, wherein one or more of the features of the tabular data modality are a time series representing diagnostic measurements for the particular patient across time.

13. The method of claim 11, wherein the time series encoder neural network is a 1-D convolutional neural network, a recurrent neural network, or a Transformer neural network.

14. The method of claim 1, wherein the language model neural network is configured to autoregressively generate an output sequence of text tokens conditioned on the input sequence, comprising:
for each position in the output sequence, generating, using the language model neural network, a probability distribution over a vocabulary of text tokens conditioned on the input sequence and any text tokens at positions preceding the position in the output sequence and selecting, using the probability distribution, a text token from the vocabulary as the text token at the position in the output sequence.

15. The method of claim 14, wherein:
the response to the query is a score that indicates a likelihood that the particular patient has a medical condition specified in the query,
processing the input sequence of tokens using a language model neural network to generate a language model output comprises processing the input sequence to determine a respective probability assigned by the language model neural network to each text token from a pre-determined output sequence of one or more text tokens conditioned on the input sequence and any tokens that precede the text token in the pre-determined output sequence, and
generating the response comprises generating the score from the respective probability assigned by the language model neural network to each text token from a pre-determined output sequence of one or more text tokens.

16. The method of claim 15, wherein generating the score comprises determining a log likelihood for the predetermined output sequence from the respective probabilities.

17. The method of claim 14, wherein:
the response to the query is a natural language response to the query,
processing the input sequence of tokens using a language model neural network to generate a language model output comprises processing the input sequence to generate one or more candidate output sequences of text tokens, and
generating the response comprises providing, as the response, one of the candidate output sequences of text tokens generated by the language model neural network.

18. A system comprising one or more computers and one or more storage devices storing instructions that are operable, when executed by the one or more computers, to cause the one or more computers to perform operations comprising:
receiving a query relating to a health of a particular patient;
obtaining multi-modal health features associated with the particular patent, the multi-modal health features comprising one or more features from each of a plurality of modalities;
generating a respective representation of each of the one or more features from each of the plurality of modalities as a respective set of one or more tokens in a natural language embedding space;
generating an input sequence of tokens that comprises one or more tokens in the natural language embedding space representing the query and the respective sets of one or more tokens in the natural language embedding space that represent each of the one or more features from each of the plurality of modalities;
processing the input sequence of tokens using a language model neural network to generate a language model output; and
generating, from the language model output, a response to the query.

19. One or more non-transitory computer storage media encoded with instructions that, when executed by one or more computers, cause the one or more computers to perform operations comprising:
receiving a query relating to a health of a particular patient;
obtaining multi-modal health features associated with the particular patent, the multi-modal health features comprising one or more features from each of a plurality of modalities;
generating a respective representation of each of the one or more features from each of the plurality of modalities as a respective set of one or more tokens in a natural language embedding space;
generating an input sequence of tokens that comprises one or more tokens in the natural language embedding space representing the query and the respective sets of one or more tokens in the natural language embedding space that represent each of the one or more features from each of the plurality of modalities;
processing the input sequence of tokens using a language model neural network to generate a language model output; and
generating, from the language model output, a response to the query.

20. The system of claim 18, wherein the multi-modal health features comprise text features, each text feature having a corresponding feature name and a feature value, and wherein generating a representation of each of the one or more features from each of the plurality of modalities as one or more tokens in the natural language embedding space comprises:
generating, from the text features, a natural language text sequence;
tokenizing the natural language sequence into a plurality of text tokens; and
mapping each text token to a respective token in the natural language embedding space using a text embedding matrix.
